(19) United States
(12) Patent Application Publication
(10) Pub. No.: US 2003/0166015 A1
(43) Pub. Date: Sep. 4, 2003

(54) Multiplexed analysis of cell-substrate interactions

(76) Inventors: Michael A. Zarowitz, San Carlos, CA (US); Oren E. Beske, Belmont, CA (US); Simon Goldbard, San Jose, CA (US)

Correspondence Address: KOLISCH HARTWELL, P.C., 520 S.W. YAMHILL STREET, SUITE 200, PORTLAND, OR, 97204, US

(21) Appl. No.: 10/382,797

(22) Filed: Mar. 5, 2003

Related U.S. Application Data
(63) Continuation-in-part of application No. 09/694,077, filed on Oct. 19, 2000.
(63) Continuation-in-part of application No. 10/120,900, filed on Apr. 10, 2002.
(60) Provisional application No. 60/362,001, filed on Mar. 5, 2002.
(60) Provisional application No. 60/129,664, filed on Apr. 15, 1999.
(60) Provisional application No. 60/170,947, filed on Dec. 15, 1999.

(30) Foreign Application Priority Data
Oct. 18, 2001 (WO) PCT/US01/51413

(51) Int. Cl. G01N 33/53; G01N 33/567

(52) U.S. Cl. 435/7.2

(57) ABSTRACT

Systems, including methods, compositions, and kits, for multiplexed analysis of interactions between cells and substrates using coded carriers.

### CROSS-REFERENCES TO PRIORITY APPLICATIONS

This application is a continuation-in-part of the following U.S. patent applications: Ser. No. 09/694,077, filed Oct. 19, 2000; and Ser. No. 10/120,900, filed Apr. 10, 2002. This application also claims the benefit under 35 U.S.C. § 119(e) of the following U.S. provisional patent application: Serial No. 60/362,001, filed Mar. 5, 2002.

U.S. patent application Ser. No. 09/694,077 is a continuation-in-part of U.S. patent application Ser. No. 09/549,070, filed Apr. 14, 2000, which in turn claims the benefit under 35 U.S.C. § 119(e) of the following U.S. provisional patent applications: Serial No. 60/129,664, filed Apr. 15, 1999; and Serial No. 60/170,947, filed Dec. 15, 1999.

U.S. patent application Ser. No. 10/120,900 claims the benefit under 35 U.S.C. § 120 of PCT Application Serial No. PCT/US01/51413, filed Oct. 18, 2001, and published as Publication No. WO 02/37944 on May 16, 2002, which in turn claims priority from the following U.S. provisional patent applications: Serial No. 60/241,714, filed Oct. 18, 2000; Serial No. 60/259,416, filed Dec. 28, 2000; Serial No. 60/293,863, filed May 24, 2001; Serial No. 60/299,267, filed Jun. 18, 2001; Serial No. 60/299,810, filed Jun. 20, 2001; Serial No. 60/307,649, filed Jul. 24, 2001; Serial No. 60/307,650, filed Jul. 24, 2001; Serial No. 60/310,540, filed Aug. 6, 2001; Serial No. 60/317,409, filed Sep. 4, 2001; Serial No. 60/318,156, filed Sep. 7,2001; and Serial No. 60/328,614, filed Oct. 10, 2001.

The above-identified U.S., PCT, and provisional patent applications are all incorporated herein by reference in their entirety for all purposes.

### CROSS-REFERENCES TO RELATED APPLICATIONS

This application incorporates by reference in their entirety for all purposes the following U.S. patent applications: Ser. No. 10/119,814, filed Apr. 9, 2002; Ser. No. 10/186,219, filed Jun. 27, 2002; Ser. No. 10/238,914, filed Sep. 9, 2002; Ser. No. 10/273,605, filed Oct. 18, 2002; Ser. No. 10/282,904, filed Oct. 28, 2002; and Ser. No. 10/282,940, filed Oct. 28, 2002.

This application also incorporates by reference in their entirety for all purposes the following U.S. provisional patent applications: Serial No. 60/362,055, filed Mar. 5, 2002; Serial No. 60/362,238, filed Mar. 5, 2002; Serial No. 60/370,313, filed Apr. 4, 2002; Serial No. 60/383,091, filed May 23, 2002; Serial No. 60/383,092, filed May 23, 2002; Serial No. 60/413,407, filed Sep. 24, 2002; Serial No. 60/413,675, filed Sep. 24, 2002; Serial No. 60/421,280, filed Oct. 25, 2002; and Serial No. 60/426,633, filed Nov. 14, 2002.

This application also incorporates by reference in their entirety for all purposes the following PCT patent applications: Serial No. PCT/US00/10181, filed Apr. 14, 2000, and published as Publication No. WO 00/63419 on Oct. 26, 2000; Serial No. PCT/US02/33350, filed Oct. 18, 2002; and Serial No. PCT/US02/34699, filed Oct. 28, 2002.

### CROSS-REFERENCES TO ADDITIONAL MATERIALS

This application also incorporates by reference in their entirety for all purposes the following U.S. Pat. Nos. 3,772,099, issued Nov. 13, 1973; No. 3,897,284, issued Jul. 29, 1975; No. 3,964,294, issued Jun. 22, 1976; No. 3,966,599, issued Jun. 29, 1976; No. 3,980,561, issued Sep. 14, 1976; No. 4,053,433, issued Oct. 11, 1977; No. 4,087,327, issued May 2, 1978; No. 4,131,064, issued Dec. 26, 1978; No. 4,197,104, issued Apr. 8, 1980; No. 4,329,393, issued May 11, 1982; No. 4,343,904, issued Aug. 10, 1982; No. 4,363,965, issued Dec. 14, 1982; No. 4,390,452, issued Jun. 28, 1983; No. 4,469,623, issued Sep. 4, 1984; No. 4,634,675, issued Jan. 6, 1987; No. 4,640,035, issued Feb. 3, 1987; No. 4,649,114, issued Mar. 10, 1987; No. 4,652,395, issued Mar. 24, 1987; No. 4,727,040, issued Feb. 23, 1988; No. 4,833,083, issued May 23, 1989; No. 4,888,294, issued Dec. 19, 1989; No. 4,906,577, issued Mar. 6, 1990; No. 4,921,792, issued May 1, 1990; No. 4,963,490, issued Oct. 16, 1990; No. 4,982,739, issued Jan. 8, 1991; No. 5,019,512, issued May 28, 1991; No. 5,079,161, issued Jan. 7, 1992; No. 5,081,036, issued Jan. 14, 1992; No. 5,096,814, issued Mar. 17, 1992; No. 5,100,783, issued Mar. 31, 1992; No. 5,100,799, issued Mar. 31, 1992; No. 5,114,853, issued May 19, 1992; No. 5,126,269, issued Jun. 30, 1992; No. 5,233,369, issued Aug. 3, 1993; No. 5,409,839, issued Apr. 25, 1995; No. 5,451,505, issued Sep. 19, 1995; No. 5,486,855, issued Jan. 23, 1996; No. 5,571,410, issued Nov. 5, 1996; No. 5,708,153, issued Jan. 13, 1998; No. 5,741,462, issued Apr. 21, 1998; No. 5,760,394, issued Jun. 2, 1998; No. 5,770,455, filed Jun. 23, 1998; No. 5,780,258, issued Jul. 14, 1998; issued Jun. 23, 1998; No. 5,817,751, issued Oct. 6, 1998; No. 5,840,485, issued Nov. 24, 1998; No. 5,961,923, issued Oct. 5, 1999; No. 5,981,180, issued Nov. 9, 1999; No. 5,989,835, issued Nov. 23, 1999; No. 5,990,479, issued Nov. 23, 1999; No. 6,025,200, issued Feb. 15, 2000; No. 6,100,026, issued Aug. 8, 2000; and No. 6,103,479, issued Aug. 15, 2000.

This application also incorporates by reference in their entirety for all purposes the following PCT Patent Applications: Serial No. PCT/IL97/00105, filed Mar. 20, 1997; Serial No. PCT/US98/21562, filed Oct. 14, 1998; Serial No. PCT/US98/22785, filed Oct. 27, 1998; Serial No. PCT/US99/00918, filed Jan. 15, 1999; Serial No. PCT/US99/01315, filed Jan. 22, 1999; Serial No. PCT/GB99/00457, filed Feb. 15, 1999; Serial No. PCT/US99/14387, filed Jun. 24, 1999; Serial No. PCT/GB99/02108, filed Jul. 2, 1999; Serial No. PCT/SE99/01836, filed Oct. 12, 1999; Serial No. PCT/US99/31022, filed Dec. 28, 1999; Serial No. PCT/US00/25457, filed Sep. 18, 2000; Serial No. PCT/US00/27121, filed Oct. 2, 2000; and Serial No. PCT/US00/41049, filed Oct. 2, 2000.

### FIELD OF THE INVENTION

The invention relates to multiplexed assays for analyzing biological systems. More particularly, the invention relates to multiplexed assays for analyzing interactions between cells and substrates using coded carriers.

### BACKGROUND OF THE INVENTION

The relative spatial distribution of cells within a multicellular organism both creates and defines surfaces or layers, termed substrates, with which the cells can interact. Substrates are fundamental to providing the structure of tissues and organs, and more generally, the entire body plan of the organism. In addition, substrates may be important for proper cellular communication, migration, nourishment, growth regulation, and individual and integrated cell function. Disruption of cell-substrate interactions may be the cause and/or effect of numerous disease states, most notably cancer.

The substrates defined by the cells themselves may be augmented by an extracellular matrix, resident in the space(s) between cells. This matrix may act as a scaffold or framework for cell attachment, cell migration, and cell communication, including sending and/or storing intercellular signals. To carry out these various functions, the extracellular matrix includes a complex mixture of structural and signaling materials such as proteins, glycoproteins, proteoglycans, and glycans, among others. This complex mixture interacts nonspecifically and/or specifically with cells and particularly cell-surface receptors to regulate many aspects of cell function, including the stimulation and/or inhibition of growth, apoptosis, differentiation, and/or cell-cell junction formation and function, among others. As a result, understanding and controlling interactions between cells and between the cells and the extracellular matrix may be critical in preventing and curing cancer, preventing or correcting birth defects, strengthening tissue, forming tissue and organs de novo, and the like.

Despite the undeniable importance of interactions between cells and substrates in many biological processes, drug screens generally have not focused on these interactions. Perhaps, the complexity of the interactions between cells and their substrates has stymied drug research in this area. Accordingly, systems are needed that allow drug candidates to be screened more efficiently for their effects on different cell-substrate interactions.

### SUMMARY OF THE INVENTION

The invention provides systems, including methods, compositions, and kits, for multiplexed analysis of interactions between cells and substrates using coded carriers.

### BRIEF DESCRIPTION OF THE DRAWINGS

FIG. 1 is a flowchart of a method for multiplexed analysis of the effect of modulators on cell-substrate interactions, in accordance with aspects of the invention.

FIG. 2 is a schematic diagram of a method for measuring interactions between test cells and different extracellular matrix materials, in accordance with aspects of the invention.

FIG. 3 is a schematic plan view of an interaction that may be measured from a well treated with a modulator in the method of FIG. 2, indicated at “3” in FIG. 2.

FIG. 4 is a schematic plan view of an alternative interaction that may be measured using a variation of the method of FIG. 2, in accordance with aspects of the invention.

FIG. 5 is a schematic plan view of still another cell-substrate interaction that may be measured using still another implementation of the method of FIG. 2, in accordance with aspects of the invention.

FIG. 6 is a schematic plan view of yet another interaction that may be measured using yet another variation of the method of FIG. 2, in accordance with aspects of the invention.

FIG. 7 is a schematic plan view of still another interaction that may be measured using still another variation of the method of FIG. 2, in accordance with aspects of the invention.

FIG. 8 is a schematic plan view of yet another interaction that may be measured using yet another variation of the method of FIG. 2, in accordance with aspects of the invention.

### DETAILED DESCRIPTION

The invention provides systems, including methods, compositions, and kits, for multiplexed analysis of cell-substrate interactions using coded carriers. Each carrier includes a substrate and a code that identifies the substrate. The substrate may be a (treated or untreated) surface of the carrier itself, or it may be some or all of an additional material that is connected to a core portion of the carrier. Thus, the substrate may be formed integrally with the carrier, or produced afterwards through some treatment and/or addition. Exemplary materials connected to a core portion of the carrier may include, but are not limited to, proteins, receptors, ligands, antibodies, synthetic polymers, extracellular matrix materials, viruses, and/or cells.

Carriers having different substrates, and thus different identifying codes, may be mixed to form an array or mixture of carriers and their substrates. The array may be tested for interaction with test cells by contacting the array with the test cells. Interactions between the test cells and individual substrates/carriers within the array may be measured. The codes of one or more of the individual carriers may be read to identify the substrate or substrates involved in one or more of the interactions. In some embodiments, the test cells and/or substrates may be exposed to one or more modulators. Accordingly, each measured interaction may be related to the substrate of a carrier and to the modulator to which the test cells and/or substrates were exposed. In addition, different substrates may be compared for their interaction with the test cells, and the effect of different modulators on the interactions may be compared. Interaction may include any effect of the substrate on a characteristic of the test cells and/or effect of the test cells on a characteristic of the substrate. Accordingly, the interaction may be binding of test cells to the substrates, and/or the size, morphology, motility, and/or distribution, among others, of the test cells relative to each other and/or the carriers after binding. Alternatively, or in addition, the interaction may be an effect on the substrate, particularly substrate cells that are included in the substrate.

The use of substrate arrays to measure cell-substrate interactions may offer a more efficient approach for studying and modifying the binding, distribution, motility, and/or other behavior of cells relative to each other or relative to their environment. Furthermore, such substrate arrays may promote the discovery of new drugs that affect cell-substrate interactions, including cell-cell interactions.

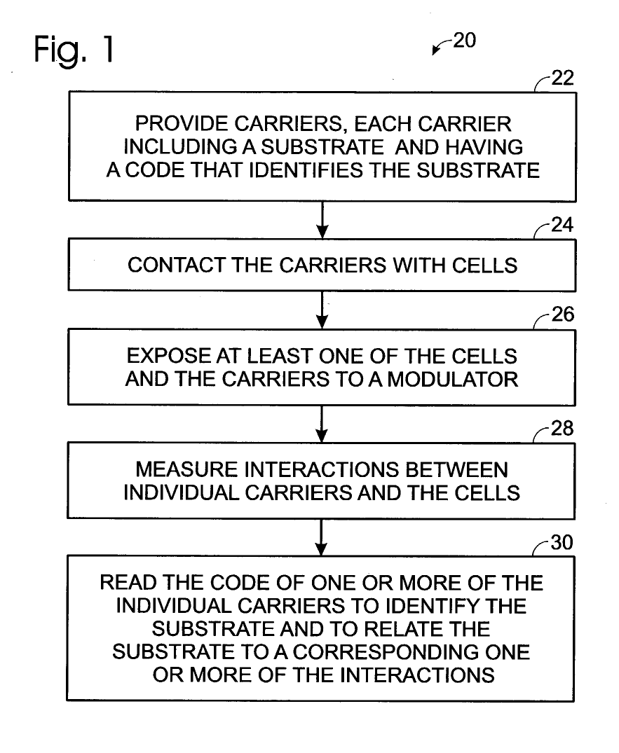

FIG. 1 shows a flowchart of a method 20 for multiplexed analysis of the effect of modulators on cell-substrate interactions, in accordance with aspects of the invention. The steps presented may be conducted in any suitable order and any suitable number of times. In addition, some of the steps, for example, exposure to a modulator, may be omitted. Typically, at least one of the steps is performed with the carrier, or a mixture of carriers, at least substantially surrounded by fluid (such as an aqueous buffer or medium).

Method 20 includes providing carriers, as shown at 22. Each carrier includes a substrate and has a code that identifies the substrate. As used herein, identifying the substrate may include identifying any suitable aspect of the substrate, such as chemical composition, method of formation, treatment, etc. The substrate may be formed of any suitable material with which test cells may interact. In some embodiments, different materials may be connected to core portions of different classes of carriers having different codes. Alternatively, carriers may be formed of different materials to provide different substrates, or the same material may be treated differently.

Carriers with different substrates (and codes) may be mixed to form a mixture. The mixture may provide carriers (and substrates) that are positioned arbitrarily relative to one another. In some embodiments, portions of the mixture may be placed in a plurality of different compartments, such as the wells of a microtiter plate (or microplate). Alternatively, mixtures of carriers may be formed in the different compartments, for example, by mixing different classes of carriers (and different substrates) in each compartment.

The carriers may be contacted with cells, as shown at 24. Such cells are termed test cells to distinguish them from any substrate cells that may at least partially form the substrate. Contacting may be initiated or facilitated by placing cells and the carriers together, that is, combining them in a shared compartment, generally in a fluid. Individual carrier classes may be placed into the compartment separately, or, as part of a mixture of carrier classes. Contacting provides an opportunity for the test cells and the carriers to interact and may occur at any time or times after the cells and carriers are placed together. Contacting may include continuous contact (binding), intermittent contact, random contact, and/or apposition between any subset of the cells and any subset of the carriers. Apposition may occur, for example, when the cells are connected to a substrate that is separate from the carriers, such as a surface of the compartment (for example, a microplate well), and the carriers placed on the cells. In some embodiments, contacting also may occur before the carriers are placed in a plurality of compartments.

The test cells and/or the carriers may be exposed to a modulator, as shown at 26. The modulator may be any chemical, biological, and/or physical agent. In some embodiments the modulator is a candidate modulator, such as a drug candidate, that is being tested for its ability to modulate interaction between the cells and the substrates. Furthermore, the modulator may modify the test cells and/or the substrates, so either or both may be exposed to the modulator. If the test cells and the carriers are placed in a plurality of compartments, the test cells and/or carriers in each compartment may be exposed to the same test candidate modulator, to provide control or corroborative information, or to a different candidate modulator, to compare the effects of different modulators.

Interactions may be measured between individual carriers and the test cells, as shown at 28. Each interaction measured may a quantitative determination of an interaction, presence/absence of any interaction, a qualitative estimate of interaction, and/or the like. The interaction may be binding of the test cells to carriers/substrates. Alternatively, or in addition, the interaction may be a change in a cell characteristic that occurs during and/or after binding or cell-substrate apposition, such as cell spreading, differentiation, movement, aggregation, change in expression of a gene, etc. The change in a cell characteristic (or the absence of a change) may be measured for test cells and/or substrate cells.

The code of one or more of the individual carriers may be read, as shown at 30. Reading the code may identify the substrate included in the one or more individual carriers. As a result, some or all of the interactions measured may be related to the substrate and to any modulator that was exposed to the test cells and/or carriers.

Further aspects of the invention are described in the following sections, including (I) coded carriers, (II) substrates, (III) arrays, (IV) cells, (V) interactions, (VI) modulators, (VII) measurement of interactions and reading codes, and (VIII) examples.

I. Coded Carriers

The coded carriers generally comprise populations of particles, distinguishable at least in part by a detectable code. Each carrier includes a substrate, either formed during production of the carrier or formed at least partially after a core portion of the carrier is produced. The carrier connects the substrate to a code. Accordingly, the code may identify the substrate and relate the identified substrate to an interaction measured between the substrate and test cells. The carriers may have any suitable composition, size, and shape consistent with an ability to perform their intended function.

Carriers may have a composition that includes glass, plastic (such as a polyacrylate), ceramic, sol-gel material, metal, protein, nucleic acid, lipid, and/or polysaccharide, among others. The material may be a solid, a gel or other porous material, and/or a combination thereof. In some embodiments, the carriers include a core portion, such as glass or plastic, among others, and a material connected to the core portion. Accordingly, the core portion may include the code and may be inanimate.

The carriers or particles generally may have any suitable size. Preferred properties are determined by the application. For example, preferred sizes may be determined in part by what the carriers are connected to and identify, with carriers preferably being at least a few times larger than the molecules, organelles, viruses, cells, and/or so on that the carriers may be connected to and support. Preferred sizes also may be determined in part by the detection method, with carriers preferably being (at least for optical detection) larger than the wavelength of light but smaller than the field of view. Preferred sizes provide carriers termed microcarriers. Microcarriers may range between about ten microns and about four millimeters in diameter (or length). Alternatively, or in addition, microcarriers may have a diameter (or length) related to the test cells that contact the carriers, with the average diameter of the microcarriers being greater than an average diameter of the test cells or between about one to fifty cell diameters, among others.

Numerous applications of the invention may be carried out in microplates, such as 96, 384, or 1536 well microplates, or similar sample holders, having a relatively high density of relatively low volume wells. In these applications, the microcarriers preferably should be small enough so that at least two or more microcarriers may be viewed in the well simultaneously. Therefore, the maximum size dimension for microcarriers sometimes may be dictated by the well dimension in a specific microplate configuration or density. Conversely, the minimum area of microcarriers preferably should be large enough to support at least one cell. Thus, microcarriers for multiplexed cellular experiments may have an area of at least about 100 square microns.

Preferred carrier geometries may include at least substantially planar, for example, in the form of a wafer or sheet, and at least substantially cylindrical. The wafer or sheet may be square, rectangular, polygonal, circular, elliptical, and/or curvilinear, among others, when viewed from the top, side, or end, and may have at least one pair of opposing surfaces that are generally parallel. In some embodiments, at least one surface provides an experimental platform for testing interaction. In some embodiments, the carriers may include one or more recesses, ridges, and/or grooves at their surfaces or may have smooth surfaces.

The code generally comprises any mechanism capable of distinguishing different carriers. The code may relate to overall features of the carriers. These features may include carrier size, shape, and/or composition. Alternatively, or in addition, the code may relate to subfeatures of the carrier. These subfeatures may be positional and/or nonpositional, meaning that the code is based on the presence, identities, amounts, and/or properties of materials at different positions in the carrier and/or at potentially the same position in the carrier, respectively. These positions may be random and/or predefined.

Exemplary positional and nonpositional codes may be optically detectable. Such codes may be formed by using materials that differ in how they generate and/or interact with light (i.e., electromagnetic radiation, particularly visible light, ultraviolet light, and infrared light), such as their absorption, fluorescence, diffraction, reflection, color (hue, saturation, and/or value), intrinsic polarization, chemiluminescence, bioluminescence, and/or any other optically distinct property or characteristic. Positional codes may be formed by positioning different amounts and/or types of materials at different positions in or on a carrier, for example, at spots, lines, concentric circles, and/or the like. These positional codes may be read by determining the identities, amounts, and/or other properties of the code materials at each code position, for example, by measuring intensity as a function of position. Nonpositional codes may be formed, for example, by using at least two different materials, potentially at the same position, where the materials differ in how they interact with light. These nonpositional codes may be read by determining the presence and/or other properties of signals from the different materials, for example, by measuring intensity as a function of wavelength for an optical code. In each case, the amounts, positions, and/or values may be relative or absolute. Moreover, different types of codes may be combined to form yet other types of codes. In some embodiments, the codes may be read directly by interrogation with light (electromagnetic radiation), without reacting or processing the carriers.

Codes may define classes of carriers. Each carrier class is defined by a different code or set of codes. Accordingly, carriers in different classes may include different substrates and/or different materials connected to a carrier core. The different substrates and/or materials are identified by the different code or set of codes.

Further aspects of coded carriers, including carriers and codes that may be suitable, are described in the patents and patent applications listed above under Cross-References, which are incorporated herein by reference, particularly the following U.S. patent applications: Ser. No. 09/694,077, filed Oct. 19, 2000; Ser. No. 10/120,900, filed Apr. 10, 2002; and Ser. No. 10/273,605, filed Oct. 18, 2002.

II. Substrates

Each coded carrier includes a substrate for testing interaction with test cells. The substrate generally comprises any exterior and/or interior surface portion or layer of the carrier that is available for interaction with test cells through contact with, or apposition to, the test cells. Substrates defined at least partially by internal surfaces or layers may be included in carriers that have a gel portion or porous configuration. In some embodiments, the substrate may comprise only a fraction of the surface of the carrier, for example, when a carrier has a nonuniform surface composition or structure. For example, portions of the carrier that provide the code and the substrate may be distinct, partially overlapping, or completely overlapping. Carriers may include distinct substrates for cell interaction based on composition, impregnation, chemical/physical modification, and or surface treatment, among others, as described below.

Different substrates may be formed based on different materials used to produce the carriers. For example, different classes of carriers may be formed throughout of different materials, such as different polymers (for example, polyethylene, polypropylene, polystyrene, or polycarbonate, among others), with each inherently presenting a different substrate for cell interaction. In some embodiments, different materials may be present only near the carrier's surface. For example, the different material may be a thin layer, such as a film or coating, connected to a surface of a carrier core. In other embodiments, the carriers may be formed as composites, such as fused or attached components, with different substrates defined by different surface regions within each of the carriers.

A material of any suitable composition may be connected to a core portion of a carrier using any suitable method. The material may define the substrate alone or in combination with the core portion. The material may be produced in vivo or vitro, may be crude or pure, may be a single component or a plurality of components, and/or may be a defined or undefined mixture. The material may include any suitable components, including one or more proteins, antibodies, receptors, ligands, cells/cell types, viruses, extracellular matrix materials, synthetic polymers, and/or the like. In exemplary embodiments, core portions of the carriers may be connected to cells or tissues that at least partially or completely define the substrates of the carriers. The material may be attached covalently or noncovalently, for example, through hydrophobic, hydrophilic, electrostatic, van der Waals, hydrogen-bonding, and/or metal-coordinating interactions, among others. In some embodiments, the material may be synthesized in situ adjacent the core portion of the carrier, for example, by covalently coupling monomer components (such as nucleotides or amino acids, among others) to form a synthetic polymer. Alternatively, the synthetic polymer may be synthesized separately and then connected to the core portion. Exemplary synthetic polymers may include a plastic, as exemplified above, poly-L-lysine, poly-D-lysine, polyethylenimine, etc.

The material that at least partially defines the substrate may be an extracellular matrix material. For example, the extracellular matrix material may be any external matrix produced by cells and deposited external to the cells, as well as any component, mixture, and/or portion thereof. An extracellular matrix component may be an extracellular matrix protein(s), a glycosaminoglcyan(s), and/or a mixture thereof. Examples of extracellular matrix proteins may include gelatin, collagen, laminin, fibronectin, entactin, vitronectin, fibrillin, elastin, and/or the like. Examples of glycosaminoglycans may include heparan sulfate, chondroitin sulfate, heparin, keratan sulfate, and/or hyaluronic acid, among others. The extracellular matrix material may be a complex mixture of compounds, such as an extracellular matrix produced by a cell(s), tissue(s), embryo(s), fetus(es), and/or organism(s), among others. In some embodiments, the substrate may include an extracellular matrix component (or components) produced by cells apposed to, and then separated from, one or more carriers.

Distinct substrates may be formed by impregnating a carrier with different compounds or materials during carrier formation. For example, carriers formed by stamping or molding may include a distinct substrate component(s) introduced during carrier formation, but only at a surface of the carriers. Further aspects of forming carriers by stamping or molding are described in the patents and patent applications listed above under Cross-References, which are incorporated herein by reference, particularly U.S. patent application Ser. No. 10/273,605, filed Oct. 18, 2002.

Different substrates may be formed or altered by chemically or physically modifying a surface of the carriers. Chemical modification may include oxidation, reduction, formation of surface charges, cyclization, chemical addition (silanization or other covalent linkage), plasma treatment, dipping, polymerizing monomers in situ, polyelectrolyte coating, and/or the like. Physical modification may include etching, scoring, and/or any other physical treatment that changes the surface topography on a cellular or subcellular scale. Further aspects of surface modification of carriers is included in the patents and patent applications listed above under Cross-References, which are incorporated herein by reference, particularly U.S. patent applications Ser. No. 10/120,900, filed Apr. 10, 2002; and Ser. No. 10/273,605, filed Oct. 18, 2002

III. Arrays

Coded carriers including different substrates and different codes (different classes of carriers) may be combined to form arrays of different substrates.

An array generally comprises any set of coded carriers having at least two or more different substrates identified by different carrier codes. The substrates may differ in any aspect, such as those described above in Section II. Thus, an array may include a set of two or more classes of carriers that include two or more different materials (such as different antibodies, different types of substrate cells, different extracellular matrix components, different synthetic polymers, etc.) and/or different physical/chemical treatments, among others.

Substrate arrays may be nonpositional, positional, or partially positional. In a nonpositional array, each substrate may be identified by the code only, independent of position. Thus, nonpositional arrays may be formed by arbitrarily and/or randomly positioning different substrates and their carriers relative to each other, that is, by forming a mixture of the carriers. In the mixture, members of different classes of carriers may have positions that are fixed or variable. In a positional array, each substrate may be identified based on the absolute and/or relative position of the carrier on which the substrate is included. An exemplary positional array is a kit including two or more classes of carriers with different substrates, such as different extracellular matrix materials. In a partially positional array, each substrate may be identified based on a combination of the code and the position of the carrier. Thus, partially positional arrays may use one code to identify two or more different substrates positioned at different positions, such as in different wells of a microplate. Further aspects of nonpositional, positional, and partially positional arrays are described in the patents and patent applications listed above under Cross-References, which are incorporated herein by reference, particularly U.S. patent application Ser. No. 10/120,900, filed Apr. 10, 2002.

IV. Cells

Coded carriers and their substrates may be combined with test cells to allow interaction between the test cells and the substrates. Any suitable number of test cells may be used. In some embodiments, the test cells are present in numerical excess over the carriers, that is, the number of test cells is greater than the number of carriers.

The test cells may comprise any membrane-bound species of interest, alive and/or dead, including eukaryotic and prokaryotic cells (and components such as organelles thereof), viruses, and vesicles. The test cells may include primary cells, established cell lines, and/or patient samples, among others. The test cells may include a mixture of diverse or similar cell types or may be a clonal set of cells. In some embodiments, the test cells may include two or more different types of cells. The different types of cells may have different origins (e.g., from different tissues, different cell lineages, different species, different individuals, different genetic backgrounds, different isolation procedures, transfection with different nucleic acids, etc).

When different types of test cells are used together, the different types may be distinguishable, for example, based on shape, expression of a cell-type restricted marker, size, etc., or may be distinguishable based on interaction with different classes of carriers.

In some embodiments, the carriers may include substrate cells that are connected to a core portion of the carriers, generally before the step of contacting. The substrate cells may be a different type of cell for each class of carrier. Alternatively, the same type of cells or mixture of cells may be connected to different classes of carriers. Accordingly, the substrate cells may at least partially define the substrates of the carriers, to allow interactions to be measured between the test cells and the substrate cells.

Suitable cells and cell populations are described in detail in the patents and patent applications listed above under Cross-References, which are incorporated herein by reference, particularly U.S. patent application Ser. No. 10/120,900, filed Apr. 10, 2002.

V. Interactions

Cell-substrate interactions may be measured during and/or after contacting a substrate array with test cells or a test cell population. Cell-substrate interactions generally comprise any physical, chemical, and/or biological interaction between the test cells and substrates of the coded carriers. An interaction is any measurable effect of the test cells on the substrates and/or of the substrates on the test cells. Thus, cell-substrate interactions may include binding of one or more of the test cells to a substrate, or detachment of one or more of the test cells from the substrate after binding, particularly in response to a modulator (see below). Alternatively, or in addition, cell-substrate interactions may include measuring any other characteristic of the test cells or substrate cells. Exemplary characteristics may include motility, morphology, and/or distribution of the test cells after binding to individual substrates, or such characteristics of the substrate cells themselves. Alternatively, or in addition, cell-substrate interactions may relate to any other characteristic of the test cells and/or substrate cells, such as a genetic, biochemical, or phenotypic modification of the cells.

Binding may include any stable or transient association between one or more of the test cells and a substrate. This binding may be mediated by any suitable interaction(s), including specific and/or nonspecific interactions. Thus, binding may be a qualitative (yes/no) analysis of whether any of the test cells adhere to a substrate (carrier). Alternatively, or in addition, binding may be a quantitative analysis of the absolute number or density of test cells or of each type of test cell bound to a carrier. The binding may be specific binding, which can be characterized by a binding coefficient. Generally, specific binding coefficients range from about 10⁻⁴ M to about 10⁻¹² M or 10⁻¹⁴ M and lower, and preferred specific binding coefficients range from about 10⁻⁵ M, 10⁻⁷ M, or 10⁻⁹ M and lower. As part of measuring binding, binding stringency may be modulated by mechanical manipulation and/or chemical/physical treatment of the carriers. For example, carriers may be manipulated mechanically by washing, stirring, sonicating, and/or moving, among others, the carriers to remove or detach test cells that are merely resting on the carriers or not attached with sufficient affinity. In some embodiments, relative binding strengths of the test cells to the substrates may be compared by measuring, repeatedly, binding of test cells after increasingly stringent mechanical manipulation. Similarly, chemical/physical treatment, such as changing media pH, ionic strength, nutrient concentration, growth factor concentration, temperature, and/or ambient atmosphere (gas concentration, composition, and/or pressure, among others), may be used to alter binding stringency.

Binding measurements may be used for various assays. Such measurements may provide comparative information about which substrates promote or inhibit test cell binding, particularly in the presence of various modulators. Such comparative information may be useful in characterizing different substrates and/or modulators. Alternatively, binding may provide information about the types of test cells that contacted the substrate array. In particular, the substrate array may provide different substrates with characterized cell-binding specificity, such as different receptor, ligands, or antibodies that interact selectively with particular cell types. Accordingly, such a substrate array may be used to identify the relative or absolute representation of different cell types in a sample of test cells, such as CD4 and CD8 cells within a blood sample. In some embodiments, such a substrate array may be used to sort different types of cells from a mixed cell population.

Detachment may include dissociation of the test cells from a substrate, after binding has been established. Cell detachment may be measured, for example, using a cell array, as described above, to test the ability of modulators to detach specific test cell populations selectively.

Cell-substrate interactions also may include motility of test cells that are bound to a substrate. Motility generally includes any aspect of cell movement, including rate, distance, direction (relative to carrier or substrate cells), frequency/rate of directional changes, and/or pattern, among others. Motility may be measured by time-lapse image acquisition, and may be facilitated by landmarks on a carrier, such as a grid or other markings. In some embodiments, motility measurement may be facilitated with a detectable material, such as fluorescently labeled, micron- or submicron-sized beads, distributed on a substrate. In these embodiments, after binding to the substrate, test cells may internalize and/or modify the detectable material, so that a resulting nonuniform pattern of the material reports a path of test cell movement. Methods and reagents that may be suitable for measuring cell motility with this strategy are available commercially from CELLOMICS, Pittsburgh, Pa.

Cell-substrate interactions also may include the morphology and/or size of the test cells after binding to a substrate. Morphology generally includes the shape of the test cells and may be related to cell identity, growth state, density on the substrate, differentiation to another cell type, health, response to growth media, and/or extent or nature of interaction between a test cell and an associated substrate. Thus, a test cell's morphology may be characterized as round, irregular, oblong, triangular, including/lacking processes or pseudopodia, and/or so on. Processes (such as axons, dendrites, and neurons) and pseudopodia further may be characterized by their number, length, transverse dimension(s), and/or branches, among others. The size of a test cell's footprint may be related to morphology and may be based on the extent to which the test cell spreads or flattens itself against the substrate. Thus, the size of test cells may indicate how test cells interact with the substrate.

Cell-substrate interactions also may include distribution of one or more of the test cells after binding to the substrate. Distribution of these test cells may be relative to each other, and/or relative to a substrate feature, such as substrate cells forming part of the substrate. Distribution of these test cells relative to each other may be a measure of cell-cell affinity relative to cell-substrate affinity. Thus, test cells may be spaced/dispersed on the substrate, arranged in tightly packed monolayers, disposed in clusters, piled up with reduced overall cell-substrate contact, and/or the like. In some embodiments, the substrate may include substrate cells that are distinguishable from the test cells. For example, substrate cells and test cells may have distinct sizes or morphologies or may be labeled differently. In these embodiments, interaction may include interaction between substrate cells and one or more test cells.

Cell-substrate interactions also may include any measurable genetic, biochemical, and/or phenotypic change in the characteristics of the test cells or substrate cells. Genetic changes include any change in the genomic sequence or content of test or substrate cells. Genetic changes may include mutations, rearrangements, transpositions, gene transfer, etc. Biochemical changes may include any change in the number, concentration, modification, subcellular distribution, or partnership, among others, of any material produced by, contained in, and/or secreted from a test or substrate cell(s). Exemplary biochemical changes may include a change in an expression level of a protein or RNA, nuclear translocation of a protein, phosphorylation of a protein, and/or the like. Phenotypic changes may include changes in growth state, differentiation, apoptosis, necrosis, position in the cell cycle, shape, electrical activity, etc. Alternatively, or in addition, substrate cells may be measured for any interaction described above, such as size, shape, distribution, motility, and/or detachment, among others.

Cell-substrate interactions also may include any measurable structural and/or functional change(s) in the substrate material itself. These changes may include the addition (e.g., deposition), removal (e.g., digestion and/or release), penetration, and/or rearrangement of substrate material(s), among others. These changes also may include the activation (e.g., turning on) and/or deactivation (e.g., turning off) of biochemically active substrate materials, including the activities of enzymes, the binding affinities of receptors and/or targets, and so on.

Further aspects of generating and measuring cell-substrate interactions (including mechanisms leading to binding and characteristics reflecting binding) are described below, in Examples 1-6, and in the patents and patent applications listed above under Cross-References, which are incorporated herein by reference, particularly U.S. patent application Ser. No. 10/120,900, filed Apr. 10, 2002.

VI. Modulators

Test cells and/or substrates/carriers may be exposed to a modulator to test the effect(s) of the modulator on interaction between the test cells and substrates. Modulators, also termed candidate modulators when the modulators are being test for an effect on interaction, generally comprise any chemical, biological, and/or physical agent or treatment that has the potential to affect a cell-substrate interaction, as described below.

Modulators may be chemical modulators, including any synthetic or naturally occurring element, molecule, polymer, complex, covalently linked molecules or polymers, noncovalently linked molecules or polymers, or heterogeneous multi-constituent assembly, or mixture thereof. Examples of chemical modulators may include compounds with known or suspected biological activity; ligands; antibodies; members of compound libraries for drug screens; single-, double- or triple-stranded, linear, branched, or circular, naturally occurring or synthetic DNA or RNA molecules; synthetic anti-sense oligonucleotides, including modified derivatives engineered for their efficacy, such as peptide nucleic acids; double stranded and/or interfering RNAs; peptides or peptide libraries; lipids; carbohydrates; and/or proteins or protein mixtures. Chemical modulators may include enzymes, especially proteases like stromelysin, tissue plasminogen activator (tPA), urokinase plasminogen activator (uPA), plasmin, elastase, gelatinase, matrilysin, collagenase, and so on, which act on the extracellular matrix, or inhibitors of these proteins (such as tissue inhibitors of metalloproteases, or TIMPs). Other exemplary chemical modulators may include hormones that interact with, or are included in, extracellular matrices, such as fibroblast growth factors, among others. Chemical modulators also may include general media composition, such as ambient gas composition, ionic strength, pH, ionic composition, divalent cation concentration (e.g., Ca²⁺ concentration), and/or nutrient mixture.

Modulators may be biological modulators. Biological modulators include biological entities, or fragments or extracts thereof. Examples of biological modulators include prokaryotic or eukaryotic cells, viruses, cell fragments, or extracts from cells, tissues, organisms, or embryos. Other biological modulators may include an expression library. Expression libraries generally comprise any library formed from cells, where members of the library express a foreign material or overexpress an endogenous material. Examples of expression libraries include phage libraries, such as phage display libraries that exhibit antibodies, receptors, or ligands; bacterial libraries in which foreign nucleic acid sequences are expressed; and eukaryotic cell libraries formed from CDNA or genomic expression libraries or other expression vectors.

Modulators may be physical modulators. Physical modulators include any environmental condition or treatment. Examples of physical modulators include heat, pressure, a gravitational field, an electric or magnetic field, light (electromagnetic radiation), and/or the like.

Test cells and/or substrates may be exposed to one or more modulators, before, during, and/or after contacting the carriers and their substrates with the test cells. In some embodiments, the test cells or substrates are pre-exposed to a modulator and then combined, with or without prior removal of the modulator. The test cells and/or substrates generally may be exposed to modulators for any suitable duration and at any suitable time point or interval during an analysis.

Modulators may modify a property of one or more of the test cells and/or substrates directly by binding to or reacting with the test cells and/or the substrates. For example, a modulator may function as a bi-functional bridging molecule that can bind to both the test cells and to one or more of the substrates, thus promoting (or inhibiting) interaction. In other cases, a modulator may function as an inhibitory molecule that binds either to a cell surface or substrate component, thus inhibiting the ability of the component to promote interaction. A modulator also may act less specifically, or nonspecifically, for example, by binding to classes or categories of groups or molecules on the cells or substrates. Thus, Ca²⁺ ions might act as a relatively nonspecific binding mediator by binding to and cross-linking negative charges on the cells and/or substrates.

Modulators may modify test cells and/or substrates indirectly by promoting a phenotypic change in the test cells and/or substrate. For example, a modulator may be an agonist or antagonist that binds to an extracellular or intracellular receptor in the test cells or in substrate cells, thereby increasing or decreasing the expression, activity, processing, and/or trafficking, among others, of an interacting component.

Further aspects of modulators are included in the patents and patent applications listed above under Cross-References, which are incorporated herein by reference, particularly U.S. patent application Ser. No. 10/120,900, filed Apr. 10, 2002.

VII. Measurement of Interactions and Reading Codes

Cell-substrate interactions may be measured and codes may be read at any time or times during an analysis. Measurement of interactions and reading codes may be performed in any order, on any number of coded carriers, and on any number of test and/or substrate cells. Moreover, these steps generally may be performed using any suitable examination site, such as a slide, a microtiter plate, or a capillary tube, and any suitable detection device, such as a microscope, a CCD array, an optical sensor, a film scanner, or a plate reader.

Interactions between the test cells and the substrates may be measured at any suitable time point(s) during and/or after contacting the coded carriers and their substrates with the test cells. The code may be read from more, the same number, or fewer carriers than the number of carriers from which an interaction is measured. Test and/or substrate cells may be visualized without staining by appropriate optical methods, such as phase-contrast microscopy or fluorescence microscopy (for example, when expressing a fluorescent protein). Alternatively, or in addition, test and/or substrate cells may be stained by incubation with dyes that label subcellular features or components, including nuclei, membranes, cytoplasm, particular proteins, particular nucleic acids, and/or the like.

The code may be read before, during, and/or after measuring the cell characteristic. Reading the code may include discerning or determining a positional and/or nonpositional code of a carrier by any suitable approach, such as optical and/or nonoptical techniques. Exemplary optical techniques include sensing light (particularly visible light, UV light, and infrared light) positionally or nonpositionally from a carrier. Exemplary nonoptical techniques may include electrical analysis of a carrier to read a nonoptical code, such as measurement of the carrier's capacitance, impedance, conductance, etc., in a positional or nonpositional fashion within the carrier. Whenever the code is read, it should be linked or linkable to the measured cell characteristic or interaction.

Exemplary methods for reading codes, measuring interactions, and labeling cells or cell components are described in more detail in the patents and patent applications listed above under Cross-References, which are incorporated herein by reference, particularly U.S. patent applications Ser. No. 09/694,077, filed Oct. 19, 2000; Ser. No. 10/120,900, filed Apr. 10, 2002; and Ser. No. 10/282,904, filed Oct. 28, 2002.

### VIII. EXAMPLES

The following examples describe selected aspects and embodiments of the invention, including methods for multiplexed analysis of cell-substrate interactions and exemplary results that may be obtained using these methods. These examples are included for illustration and are not intended to limit or define the entire scope of the invention.

### Example 1

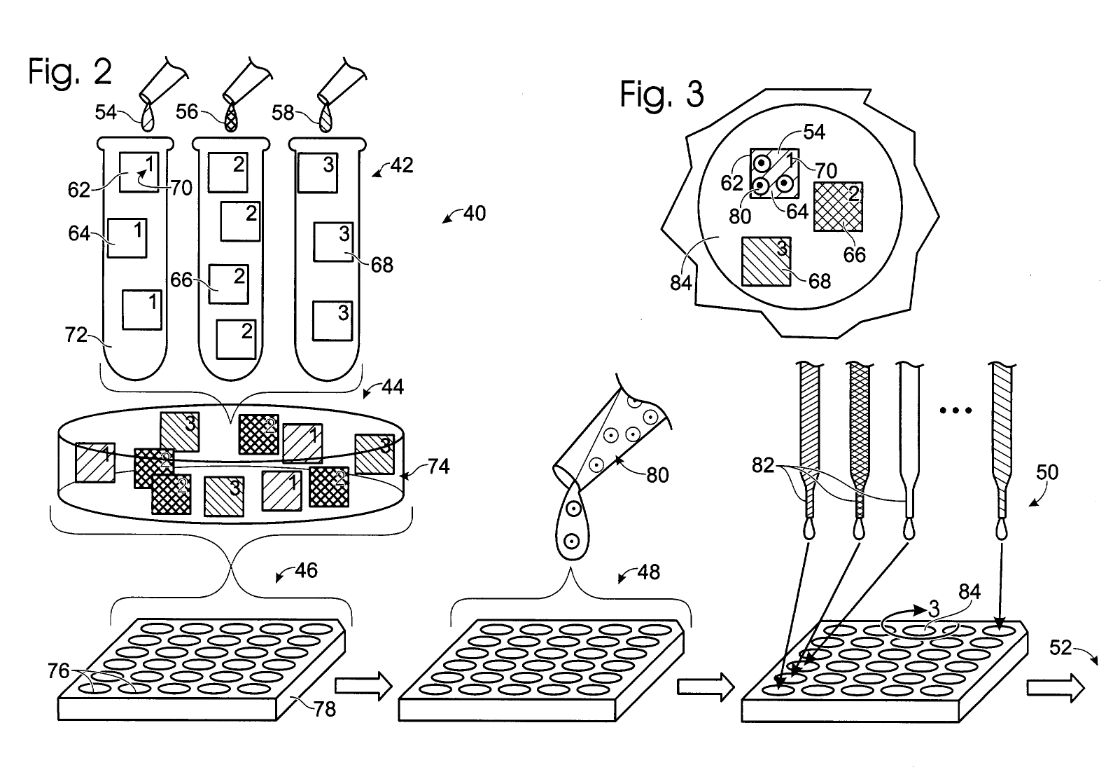

This example describes a method for multiplexed analysis of the binding of cells to different extracellular matrix materials that are included in coded carriers, and also shows an interaction that may be measured using the method; see FIGS. 2-3.

FIG. 2 schematically depicts steps that may be included in method 40. A step of connecting materials to carriers is shown at 42, a step of mixing carriers at 44, a step of placing carriers at 46, a step of contacting with test cells at 48, a step of exposing to modulators at 50, and a step of measuring and reading at 52.

The step of connecting 42 may be conducted by combining extracellular matrix materials 54, 56, 58 with coded carriers 62 of different classes 64, 66, 68, respectively. Each carrier 62 may be considered a core portion including a code 70 that identifies the corresponding connected matrix material. Connecting may be conducted in distinct compartments, such as tubes 72. Alternatively, connecting may be conducted during the production of coded carriers 62, for example, if each carrier is formed of a different material. In other embodiments, carriers 62 may be modified also or alternatively by connection to substrate cells, antibodies, ligands, synthetic polymers and/or the like.

The step of mixing 44 the coded carriers may be performed by mixing different classes of coded carriers, generally after the step of connecting 42. Here, coded carriers 62 are combined to produce a mixture or positionally unconstrained array 74 of carriers including different extracellular matrix materials.

The step of placing 46 may be performed before, during, or after the step of mixing. Here, array 74 is placed in a plurality of compartments or wells 76 provided by a microtiter plate 78. Accordingly, each well may hold a similar subarray of coded carriers and their extracellular matrix materials and may serve as an assay site for analysis of interactions. Differences in the subarrays may reflect variations produced as array 74 is sampled repeatedly or purposeful changes in the classes and number of coded carriers placed in the wells.

The step of contacting 48 may be initiated by adding test cells 80 to a compartment that holds the coded carriers. Here, test cells 80 are added to the carriers after the coded carriers have been placed at assay sites by dispensing the test cells to each well 76. However, in other embodiments, test cells 80 also may contact the coded carriers before the coded carriers are placed at assay sites, such as before the step of mixing 44 and/or before the step of placing 46. In some embodiments, test cells 80 may be dispensed to wells 76 before the coded carriers and may attach to a surface of the wells or remain unattached. Test cells 80 may be a single type of cells or a set of different types of cells. The different types of cells may contact the coded carriers at each assay site, or different types of cells may contact the carriers at different assay sites.

The step of exposing 50 may be performed by combining test cells 80 with modulators 82. Cells alone or combined with coded carriers may be exposed to modulators 82 at any stage of method 40. For example, coded carriers may be exposed to modulators before the step of contacting 48 with cells. Such an order of exposure to modulators may be suitable, for example, in alternative embodiments that connect coded carriers to substrate cells during the step of connecting 42. Alternatively, or in addition, test cells 80 may be exposed to modulators before these test cells contact the coded carriers, or both test cells 80 and the coded carriers may be exposed after they are combined, as shown here.

The step of measuring and reading 52 may be performed in method 40. Measuring provides a determination of interactions between test cells 80 and individual coded carriers. Reading the code of coded carriers identifies the extracellular matrix material or other material connected to individual coded carriers. Measuring and reading may be performed at any suitable time or times during method 40. For example, measuring and reading may be conducted after exposure to modulators, as shown here, so that any effect of the modulators may be determined. Alternatively, or in addition, measuring and reading may be performed before and after exposure to modulators or repeatedly to measure time-based changes in interaction.

FIG. 3 shows a schematic plan view of an interaction that may be measured between cells and coded carriers from an assay site 84 in FIG. 2, indicated at “3” in FIG. 2. Each coded carrier 62 is measured for binding of test cells 80 to the carrier. Here, test cells 80 bind preferentially to the substrate that includes extracellular matrix material 54, which is identified by reading the code, shown at 70, from the corresponding carrier.

### Example 2

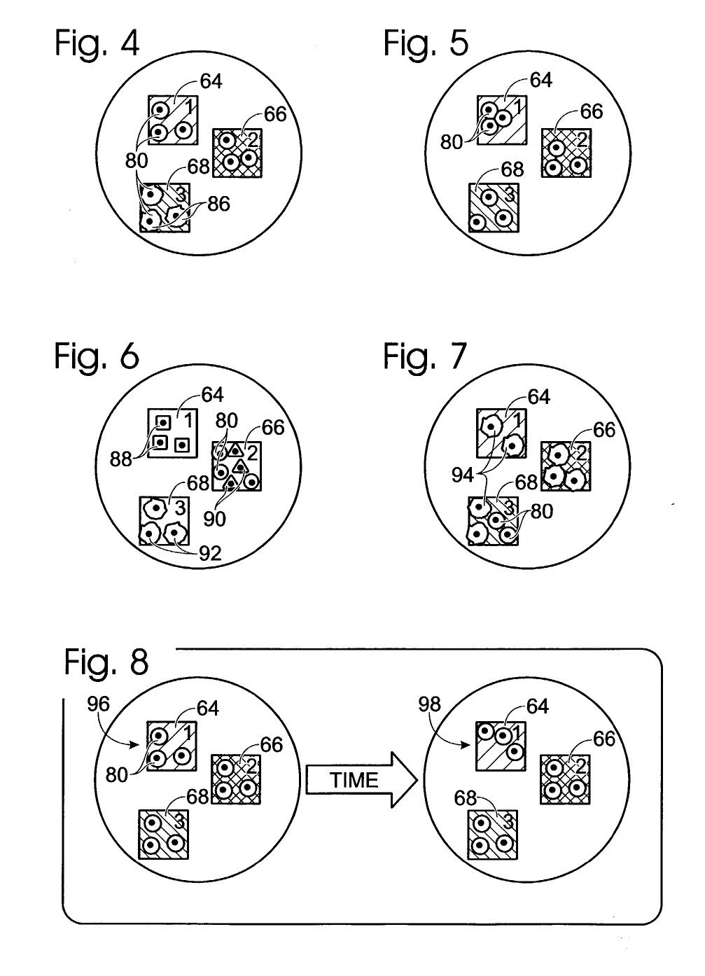

This example describes an interaction that may be measured using a method for multiplexed analysis of cell morphology on different substrates provided by coded carriers; see FIGS. 2 and 4.

Method 40 of FIG. 2 may be modified by measuring a different interaction between test cells 80 and coded carriers. Here, the number of test cells 80 bound to each class of carrier is not affected substantially by the class of carrier. However, class 68 of carriers (code “3”) presents distinctly shaped cells 86 that are more flattened or spread out than on carrier classes 64, 66. Accordingly the measured interaction may be the size of the cells (such as the carrier area occupied by some or all of the cells bound to the carrier), their shape, and/or their resistance to detachment from the carriers, among others.

### Example 3

This example describes an interaction that may be measured using a method for multiplexed analysis of cell distribution on different substrates provided by coded carriers; see FIGS. 2 and 5.

Method 40 of FIG. 2 may be modified by measuring a different interaction between test cells 80 and coded carriers. Here, the number of test cells 80 bound to each class of carrier is not affected substantially by the class of carrier. However, the spacing between cells may be distinct. For example, test cells 80 bound to carrier class 64 are more tightly clustered than on carrier classes 66, 68. Accordingly, the measured interaction may be average spacing of cells, closest spacing of cells, number of cells in a cluster, etc.

### Example 4

This example describes an interaction that may be measured using a method for multiplexed analysis of cell-cell interactions with coded carriers; see FIGS. 2 and 6.

Method 40 of FIG. 2 may be modified by connecting substrate cells 88, 90, 92 to each class of carrier 64, 66, 68, respectively. The substrate cells may be different types of cells, so that the carrier code identifies each type of cell. The substrate cells may be connected in place of extracellular matrix materials 54, 56, 58, as shown here, or in addition to these components.

The interaction measured may be binding of test cells 80 to each coded carrier. For example, here, test cells 80 bind more efficiently to carrier class 66, which includes substrate cells 90. The interaction measured may be detectable contact with test cells 80 and the substrate cells, proximity between test cells 80 and the substrate cells, or number of test cells 80 apposed to each carrier class, among others.

### Example 5

This example describes an interaction that may be measured using a method for multiplexed analysis of interactions between test cells and substrate cells apposed to different extracellular matrix materials; see FIGS. 2 and 7.

Method 40 of FIG. 2 may be modified by connecting extracellular matrix materials 54, 56, 58 to carrier classes 64, 66, 68, respectively, and substrate cells 94 to all carriers. The same type of substrate cells 94 may be connected to each carrier class, or different types may be connected, as in Example 4.

Any suitable interaction may be measured between test cells 80 and the carriers. Here, binding of test cells 80 is measured, with test cells 80 preferentially binding to carrier class 68. However, in other embodiments, the measured interaction may be proximity between test cells 80 and substrate cells 94, or any other interaction described herein.

### Example 6

This example describes an interaction that may be measured using a method for multiplexed analysis of cell motility with coded carriers; see FIGS. 2 and 8.

Method 40 of FIG. 2 may be modified by measuring movement of test cells 80 on the coded carriers. Such movement or motility may be measured by any suitable method, including time-lapse photography, a movement indicator material disposed on the carriers, etc. Here, test cells 80 of carrier class 64 show a greater change in distribution over time, shown at 96 and 98, than the test cells disposed on other carrier classes 66, 68.

### Example 7

This example presents an alternative description of selected aspects of the invention.

Substrate arrays may be prepared as follows. Carriers of different classes may be prepared by coating each of the classes with a different “binding matrix.” For example, such matrices could be fibronectin, collagen, various laminins, etc. A population of a mixture of these classes of carriers may be deposited into the wells of a microplate. Generally, these carriers do not contain any cells at this point (only the binding matrix). However, a modification is described below in which cells also may be pre-seeded onto the carriers.

An aliquot of a particular type of cell(s) may be added to each well, as well as a chemical to be tested. The assay then may determine if the added chemical inhibits or enhances binding, spreading, etc. of the cells onto each type of matrix. The readout may be as simple as DAPI-staining the cells and counting the number of cells bound to each type of matrix. Another readout may be a cytoplasm stain to determine the extent to which the bound cells have spread out (their morphology) on the matrix.

Such a test may be useful for discovering chemicals that affect the binding and subsequent morphology of cells to various biologically relevant matrices. In particular, this test may be used to study the binding of metastatic cells to a new site, or the development of particular tissues via the correct juxtaposition of different cells types.

A modification of the strategy described above may employ substrate cells to form at least part of the substrate. In this modification, the carriers may be pre-seeded with a particular type of cell, in addition to the binding matrix. This pre-seeded cell type may be known or suspected to interact with the cell type that is added separately to the wells (see above). In this type of experiment, the assay may measure the ability of added cells to interact with the pre-seeded cells on the carriers, depending on the matrix used on the carrier and the chemical added to the well.

This approach also may be used to identify surfaces/substrates that inhibit or promote the binding of cells and/or molecules. Such an approach may be of particular interest to developers of medical devices for use within the body. Classes of carriers may be prepared with various coating of materials or molecules connected to the carriers, either single materials/molecules or composites or mixtures. Aliquots of these classes may be mixed, subjected to further treatment if necessary either before or after mixing, and challenged with various cell types or molecules under various conditions. Binding or lack of binding of these entities to the particular classes (and therefore, particular surfaces) then may be determined. Examples of non-biological materials are polymers. Examples of methods may include plasma treatments, dipping, polymerizing monomers in place, and polyelectrolyte coating (for example, Mendelsohn, J. D., Yang, S. Y., Hiller, J. A., Hochbaum, A. I., and Rubner, M. F. (2003) “Rational design of cytophilic and cytophobic polyelectrolyte multilayer thin films,” Biomacromolecules 4: 96-103).

### Example 8

This example describes selected embodiments of the invention, presented as a series of indexed paragraphs.

1. A composition for multiplexed analysis of cell-substrate interactions, comprising a set of cell carriers, each cell carrier including a code and a substrate adapted to test cell interaction, wherein the substrate has an aspect identified by the code, the aspect being distinct for at least one carrier of the set.

2. The composition of claim 1, wherein the set includes at least three carriers for which the code is distinguishable, the substrate aspect identified by each of the distinguishable codes being distinct.

3. The composition of claim 1, wherein each carrier of the set includes a support structure having a surface, the surface being associated with a material that is distinct from the support structure and that at least partially forms the substrate.

4. The composition of claim 3, wherein the material includes at least one of an extracellular matrix component and a cell.

5. The composition of claim 3, wherein the support structure at least substantially includes glass.

6. The composition of claim 1, wherein each carrier of the set includes a surface associated with substrate cells, the substrate cells at least partially forming the substrate.

7. The composition of claim 1, wherein the aspect is at least one of the group consisting of type of cells that contribute to the substrate, extracellular matrix component associated with the substrate, carrier composition, carrier surface chemistry, and carrier pretreatment.

8. A method of measuring cell-substrate interactions using (1) a cell population, and (2) a set of carriers, each carrier including a code and a substrate, wherein the substrate has an aspect identified by the code, the method comprising (a) combining the set of carriers with the cell population, (b) measuring interaction between the cell population and the carriers of the set, if any, and (c) reading the code of at least one carrier of the set, thereby identifying the substrate aspect corresponding to the interaction.

9. The method of claim 8, wherein the step of measuring interaction includes detecting cells of the cell population bound to the carriers of the set.

10. The method of claim 8, wherein the aspect differs between at least two carriers of the set.

11. The method of claim 10, wherein the aspect differs between three or more carriers of the set.

12. The method of claim 8, wherein the aspect is included in plural carriers of the set, the set having two or more classes of carriers for which the aspect is distinct.

13. The method of claim 8, wherein the step of measuring interaction includes measuring movement of an individual cell of the population relative to the substrate.

14. The method of claim 8, wherein the step of measuring interaction includes measuring size of a footprint formed by a cell of the population bound to the substrate.

15. The method of claim 8, wherein at least some of the substrates include substrate cells associated with the set of carriers before combining the set of carriers with the cell population.

16. The method of claim 15, wherein the code identifies the substrate cells.

17. The method of claim 15, wherein the step of measuring includes determining proximity of at least one cell of the cell population relative to at least one of the associated substrate cells.

18. The method of claim 8, wherein the step of measuring includes determining cell morphology for a member cell of the cell population bound to one of the carriers of the set.

19. The method of claim 8, further comprising exposing the cell population or the set of carriers to at least one modulator to relate activity of the at least modulator to the measured interaction.

20. The method of claim 19, wherein the cell population and the set of carriers is exposed to the at least one modulator.

21. The method of claim 19, wherein the at least one modulator is selected from the group consisting of nucleic acids, small compounds, drug candidates, peptides, proteins, carbohydrates, and lipids.

22. The method of claim 19, the cell population comprising plural distinct cell populations, wherein the step of combining associates each of the plural distinct cell populations with a subset of carriers of the set, and wherein the step of reading the code identifies one or more of the plural distinct cell populations that is associated with the carriers of the subset.

23. The method of claim 22, the subset being associated with the one or more distinct cell populations separately from other carriers of the set, wherein the step of combining includes mixing the subset with the other carriers after associating.

24. The method of claim 22, wherein the interaction is selected from the group consisting of cell morphology, cell detachment, and cell motility.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

We claim:

1. A method of measuring cell-substrate interactions, comprising:
contacting a mixture of carriers with cells, the mixture having at least two carrier classes, each carrier class including a different substrate and having a code that identifies the different substrate, the cells having an average diameter, the carriers having an average length, the average length of the carriers being greater than the average diameter of the cells;
measuring an interaction between the cells and one or more of the carriers; and
reading the code of at least one of the one or more carriers to relate the interaction to the different substrate identified by the code.

2. The method of claim 1, wherein each carrier class includes one or more carriers.

3. The method of claim 2, wherein the step of contacting includes placing the cells and at least one carrier of each carrier class together in a shared compartment.

4. The method of claim 1, wherein the carriers each include a core portion and a material connected to the core portion, the material being included in the different substrate and being different for each carrier class.

5. The method of claim 1, wherein the material is selected from at least one of the groups consisting of antibodies, ligands, receptors, synthetic polymers, extracellular matrix materials, viruses, and cells.

6. The method of claim 5, wherein the material for each carrier class includes at least one different type of cell.

7. The method of claim 1, wherein the interaction measured is at least substantially no detectable interaction.

8. The method of claim 1, wherein the interaction measured is binding of the cells to each of the one or more carriers.

9. The method of claim 1, wherein the step of contacting connects at least a subset of the cells to the carriers, the interaction measured being at least one of distribution, morphology, size, and motility of one or more cells of the subset.

10. The method of claim 1, wherein the carriers are substantially planar.

11. The method of claim 1, wherein the code is positional.

12. The method of claim 1, wherein the carriers are microcarriers.

13. The method of claim 1, wherein the code is detectable directly by interrogation with light.

14. The method of claim 1, wherein the carriers include a surface, each different substrate being produced by a different treatment of the surface.

15. A method of measuring cell-substrate interactions, comprising:
contacting a mixture of carriers with cells, the mixture having at least two carrier classes, each carrier class including a different substrate and having a code that identifies the different substrate, the cells being present in numerical excess over the carriers;
measuring an interaction between the cells and one or more of the carriers; and
reading the code of at least one of the one or more carriers to relate the interaction to the different substrate identified by the code.

16. The method of claim 15, wherein the interaction measured is binding of the cells to each of the one or more carriers.

17. The method of claim 15, wherein the step of contacting connects at least a subset of the cells to the carriers, the interaction measured being at least one of distribution, morphology, size, and motility of one or more cells of the subset.

18. The method of claim 15, wherein the carriers are substantially planar.

19. The method of claim 15, wherein the code is positional.

20. The method of claim 15, wherein the code is detectable directly by interrogation with light.

21. A method of screening for modulators of cell-substrate interactions, comprising:
contacting a mixture of carriers with cells, the mixture having at least two carrier classes, each carrier class including a different substrate and having a code that identifies the different substrate, the cells having an average diameter, the carriers having an average length, the average length of the carriers being greater than the average diameter of the cells;
exposing at least one of the mixture and the cells to a candidate modulator;
measuring an interaction between the cells and one or more of the carriers; and
reading the code of at least one of the one or more carriers to relate the interaction to the different substrate identified by the code and thus to any effect of the candidate modulator on the interaction of the cells with such different substrate.

22. The method of claim 21, wherein at least the steps of exposing and measuring are performed a plurality of times, the candidate modulator being different each of the times.

23. The method of claim 22, wherein at least the step of exposing is performed in a different well of a microplate each of the times.

24. The method of claim 21, wherein the effect is an increase in interaction between the cells and the different substrate with exposure to the candidate modulator relative to without such exposure.

25. The method of claim 21, wherein the carriers each include a core portion and a material connected to the core portion, the material being included in the different substrate and being different for each carrier class, the material being selected from at least one of the groups consisting of antibodies, ligands, receptors, synthetic polymers, extracellular matrix materials, viruses, and cells.

26. The method of claim 25, wherein the material includes at least one of a different type of cells and a different extracellular matrix material.

27. The method of claim 21, wherein the interaction measured is at least substantially no detectable interaction.

28. The method of claim 21, wherein the step of contacting connects at least a subset of the cells to the carriers, the interaction measured being at least one of cell size, distribution, morphology, and motility of one or more cells of the subset.

29. A kit for multiplexed analysis of cell-substrate interactions, comprising:
a set of carriers, the set including at least two carriers classes, each carrier class having a different code and including a different extracellular matrix material, the different extracellular matrix material being identified by the different code.

30. The kit of claim 29, wherein each different extracellular matrix material includes one or more extracellular matrix components.

31. The kit of claim 29, further comprising at least one additional class of carriers, the additional class including a synthetic polymer and having a code that identifies the synthetic polymer.

32. The kit of claim 29, wherein each different extracellular matrix material is isolated from cells by apposing the cells to carriers of the corresponding class and then at least substantially separating the cells from the carriers of such class.

33. A system for measuring cell-substrate interactions, comprising:
means for contacting a mixture of carriers with cells, the mixture having at least two carrier classes, each carrier class including a different substrate and having a code that identifies the different substrate, the cells having an average diameter, the carriers having an average length, the average length of the carriers being greater than the average diameter of the cells;
means for measuring an interaction between the cells and one or more of the carriers; and
means for reading the code of at least one of the one or more carriers to relate the interaction to the different substrate identified by the code.
